(19) United States
(12) Patent Application Publication
(10) Pub. No.: US 2022/0417453 A1
(43) Pub. Date: Dec. 29, 2022

(54) SYSTEM FOR A THERMAL MONITORING SECURITY CAMERA

(71) Applicants: Brad Ritti, Winter Garden, FL (US); Mark Maycock, Kissimmee, FL (US); Any Lai, Orlando, FL (US); Melanie Linscott, Winter Park, FL (US)

(72) Inventors: Brad Ritti, Winter Garden, FL (US); Mark Maycock, Kissimmee, FL (US); Any Lai, Orlando, FL (US); Melanie Linscott, Winter Park, FL (US)

(21) Appl. No.: 17/853,628

(22) Filed: Jun. 29, 2022

Related U.S. Application Data
(60) Provisional application No. 63/216,030, filed on Jun. 29, 2021.

(51) Int. Cl. H04N 5/33; A61B 5/01; G01J 5/00; G06T 7/73; G06V 40/16

(52) U.S. Cl. CPC H04N 5/33; A61B 5/015; G01J 5/0025; G06T 7/73; G06V 40/171; G01J 2005/0077; G06T 2207/10048; G06T 2207/30201

(57) ABSTRACT

A system for a thermal monitoring security camera is disclosed, including at least one camera positioned to monitor a facility. An image processing module receives imagery from the at least one camera and analyzes the imagery to determine, via a facial recognition module, an identity of an individual within the imagery and the presence or absence of a mask on the face of the individual. At least one thermal camera monitors the temperatures of each individual sensed by the thermal camera. An alert module transmits an alert if the temperature is above a threshold temperature. A security system provides access or prevent access to the facility.

### CROSS-REFERENCE TO RELATED APPLICATIONS

The present application claims priority to U.S. Provisional Patent Application No. 63/216,030 filed Jun. 29, 2021, entitled “SYSTEM FOR A THERMAL MONITORING SECURITY CAMERA” which is hereby incorporated by reference in its entirety.

### TECHNICAL FIELD

The embodiments generally relate to security systems and more particularly to network-connected thermal monitoring security cameras to surveil persons entering a facility and enforce temperature, mask, and security policies.

### BACKGROUND

Thermal imaging cameras create an image using infrared radiation and are often used in security systems. These systems integrate sensing, signal extraction, processing, and comprehension processes to identify objects in the camera's field of vision. Their use is beneficial in security systems as they are able to see objects in the dark, as well as through smoke or other deterrents. Humans can be identified by thermal cameras due to the body's natural ability to produce a heat signature. This allows automated systems to detect humans using a thermal imaging camera.

Due to the prevalence of COVID-19 and other pathogens, identifying infected individuals is an important step in reducing the spread of pathogens. Many businesses have employed thermometers to measure the temperature of people entering their premise. Those having a temperature above a threshold may then be asked to not enter until their temperature returns to normal. Further, many businesses have implemented a mask policy which requires those entering the premise to wear a mask. Enforcement is often performed by employees or security personnel to ensure compliance. While effective, enforcement requires vigilance and willingness to enforce the policy which may lead to confrontation.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments provided herein relate to a system for a thermal monitoring security camera, including at least one camera positioned to monitor a facility. An image processing module receives imagery from the at least one camera and analyzes the imagery to determine, via a facial recognition module, an identity of an individual within the imagery and the presence or absence of a mask on the face of the individual. At least one thermal camera monitors the temperatures of each individual sensed by the thermal camera. An alert module transmits an alert if the temperature is above a threshold temperature. A security system provides access or prevent access to the facility.

The system utilizes thermal cameras and video cameras positioned to view the entry points and surrounding environment of the facility. The system is utilized by the facility to automate security processes, reduce the likelihood of an infectious outbreak, to increase compliance to mask policies, and as a means for recording and monitoring individuals entering and exiting the facility.

In one aspect, an application server is in operable communication with a network to host an application system having a user interface for permitting a user to input a plurality of alert settings.

In one aspect, the alert settings include a list of permitted individuals, a threshold temperature, and a door lock trigger operated by the security system, etc.

In one aspect, the alert is audible and or visible.

In one aspect, the user inputs a face profile comprising an image utilized by the facial recognition module to compare the image to imagery captured by the camera.

In one aspect, a monitoring interface includes a video feed and a thermal imagery feed. The monitoring interface may also include a listing of individuals identified by the facial recognition module.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the system infrastructure and connected network, according to some embodiments;

FIG. 2 illustrates a block diagram of the application system for the thermal camera monitoring system, according to some embodiments;

FIG. 3 illustrates a block diagram of the server engine and modules associated with the thermal camera monitoring system, according to some embodiments;

FIG. 4 illustrates a screenshot of the monitoring interface, according to some embodiments;

FIG. 5 illustrates a screenshot of the face profile interface, according to some embodiments;

FIG. 6 illustrates a screenshot of the alert settings interface, according to some embodiments; and

FIG. 7 illustrates a screenshot of the alert settings interface, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, and/or computer program product at any possible technical detail level of integration. A computer program product can include, among other things, a computer-readable storage medium having computer-readable program instructions thereon for causing a processor to carry out aspects of the present disclosure.

In general, the embodiments provided herein relate to a system for a thermal monitoring security camera which can be employed at a facility to monitor the temperature of those entering the facility. The system may also monitor and enforce mask policies to ensure that those entering the facility are properly wearing a mask prior to entrance. The system utilizes thermal cameras positioned to view the entry points and surrounding environment of the facility to identify individuals, determine if the individual is potentially experiencing an infection, and determine if the individual is adhering to facility policies (e.g., determining if the individual is wearing a mask). Each determination is made prior to the individual passing by or through an area covered with the camera, with or without gaining access to the entry point.

In some embodiments, the system includes a security system in operable communication with door locks or other means of selectively controlling access to the facility. For example, the system may determine that a person approaching the facility has an acceptable temperature and is appropriately wearing a mask, resulting in the doorway being unlocked. Each data point may be associated with an individual via a facial recognition module configured to identify the individual entering the facility.

As used herein, the term “facility” refers to any premise to which access can be selectively approved or denied. For example, the facility may be a school or business. In the example of a school, the system may be used to identify, via the facial recognition module, students entering the school storing a record of each student's body temperature to determine if the student is ill. Further, the system may monitor each student's adherence to the school's mask policy.

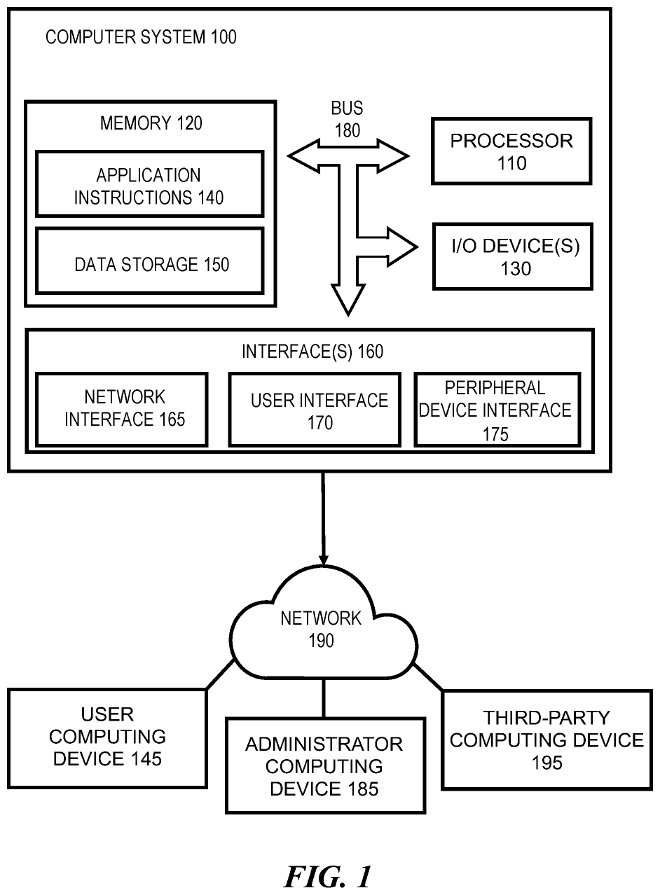

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., C, C++, C#, JAVA, JAVASCRIPT, PERL, etc.).

In this disclosure, terms “store,” “storage,” “data store,” “data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized to establish credentials, create a user profile, track metrics provided by the system, and otherwise interact with the various functionalities of the system. The third-party computing device 195 may be utilized by third parties to receive communications from the user computing device and/or administrative computing device 185.

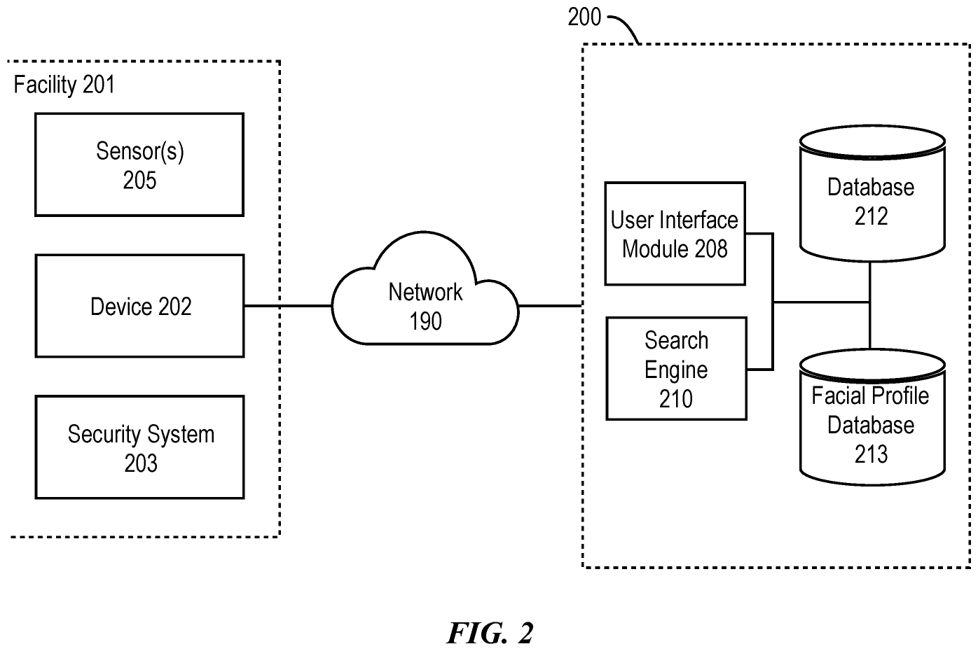

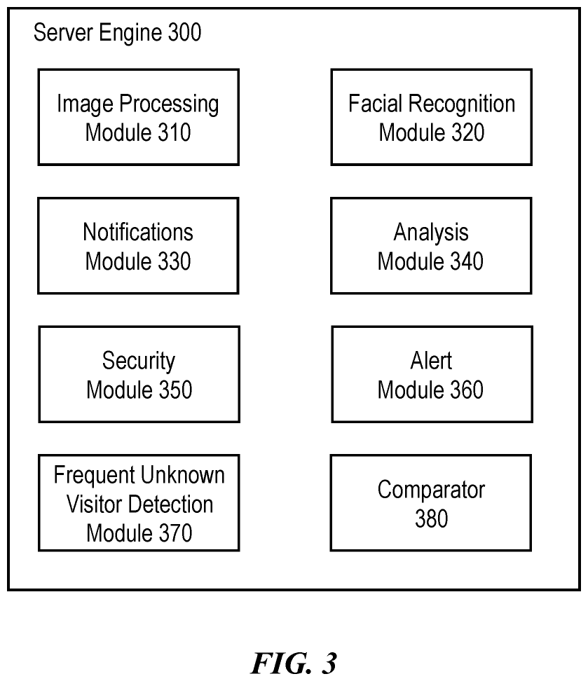

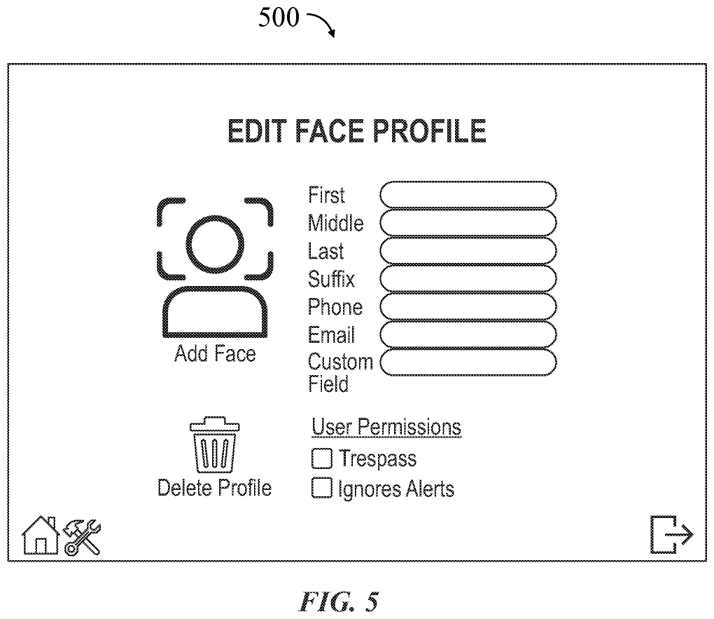

FIG. 2 illustrates a block diagram of the application system 200 in operable communication with a device 202 via a remote storage 130. A facility 201 is in communication with the device 202. Sensor(s) 205 are positioned to monitor entry points or other areas of the facility 201. A security system allows for the autonomous and/or manual locking and unlocking of entry points and/or alarm initiation upon the breach of a policy established by a user of the system. For example, the policy may be defined as a range of body temperatures which are permitted to enter the facility. In another example, the policy may be a mask policy which requires individuals entering the facility to be wearing a mask. The application system includes a user interface module 208 to display information stored in database 212. The information stored in the database may include user information, facility information, preferences, policy information, sensor data, and the like which may be searched via search engine 210. A facial profile database 213 stores facial imagery or other biometric data input in the facial profile interface (see FIG. 5). The facial profile database 213 is in communication with the facial recognition module 320 and comparator 380 (see FIG. 3).

In some embodiments, the sensor(s) 205 may include thermal imaging cameras, video cameras, microphones, and other sensors commonly associated with monitoring and security systems.

FIG. 3 illustrates a block diagram of the server engine 300 and modules including an image processing module 310, facial recognition module 320, notifications module 330, analysis module 340, security module 350, alert module 360, a frequent unknown visitor detection module 370, comparator 380. The image processing module 310 receives imagery from the sensors and processes the images to identify features contained in the images. For example, the image processing module 310 identifies individuals in the sensors sensory field (e.g., the field of view of a thermal camera). Imagery is characterized and transmitted to an analysis module 340 which analyzes features extracted from the imagery. Features may include temperature, identity, presence or absence of a mask, etc. The facial recognition module 320 utilizes a comparator to compare the facial features of the individual to stored imagery of individuals. The notifications module 330 provides notifications in response to actions performed within the system, or in response to events occurring which are relevant to the security of the facility. The security module 350 is in operable communication with the facilities security devices such as door locks, alarms, etc. For example, the security module 350 may lock a door if an approaching individual has a temperature exceeding the threshold. The alert module 360 transmits an alert to the device and/or to the security system in response to an event. For example, the alert module 360 transmits an alarm signal if unauthorized entry was made by an individual.

In some embodiments, a frequent unknown visitor detection module 370 is in operable communication with the thermal camera monitoring system. When the camera software detects an unknown user, a unique identifier is assigned to the image, and it is stored in the computer memory. If that user is detected multiple times throughout the course of a set time-frame, the occurrence is logged and the results of all users frequenting the facility without registration are displayed for examination by an authorized user. If the set time-frame expires without the unknown person returning the record is deleted from the database. Actions based on occurrence frequency (ie; barring entry upon 10 occurrences) can be enabled in the application.

In some embodiments, the comparator 380 is in communication with the facial recognition module 320 to compare imagery received from the camera and compare the imagery to face profiles stored in a face profile database.

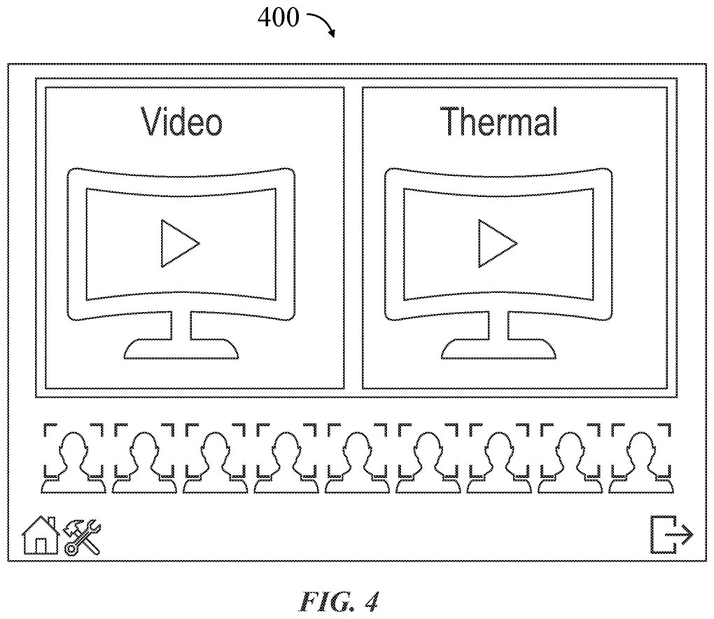

FIG. 4 illustrates a screenshot of the monitoring interface 400 which is displayed via the user interface module on the device. The monitoring interface 400 allows facility personnel to monitor sensor feeds such as those received from the thermal camera and/or video camera. The monitoring interface may display a listing of recent individuals identified by the facial recognition module. In such, the monitoring interface may be used to view who has entered and/or exited the facility.

In some embodiments, the monitoring interface 400 may include a time stamp to indicate a date and time at which each individual entered the facility. In some examples, the time stamp and recognition of the individual may be used as a means of taking attendance, or as a means of clocking in for a work day.

FIG. 5 illustrates a screenshot of the face profile interface 500 wherein a user can add or edit a face profile which is stored in the system. In use, the face profile interface 500 allows the user to create a listing of approved individuals may enter the facility. User permissions may be established such that the individual is granted access permissions once identified by the system. For example, once the user is identified, they may be able to enter and exit the facility at specific times each day. To add a user profile, an image of the individual is added which can be used by the facial recognition module to identify the individual when entering or exiting the facility. Personal information such as a name and contact information may be input to complete the creation of the face profile. The image of the individuals face is stored in the face profile database.

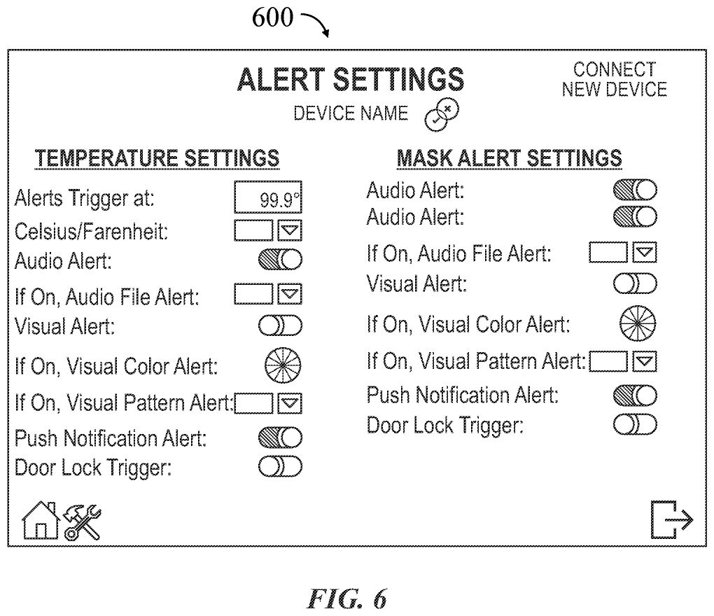

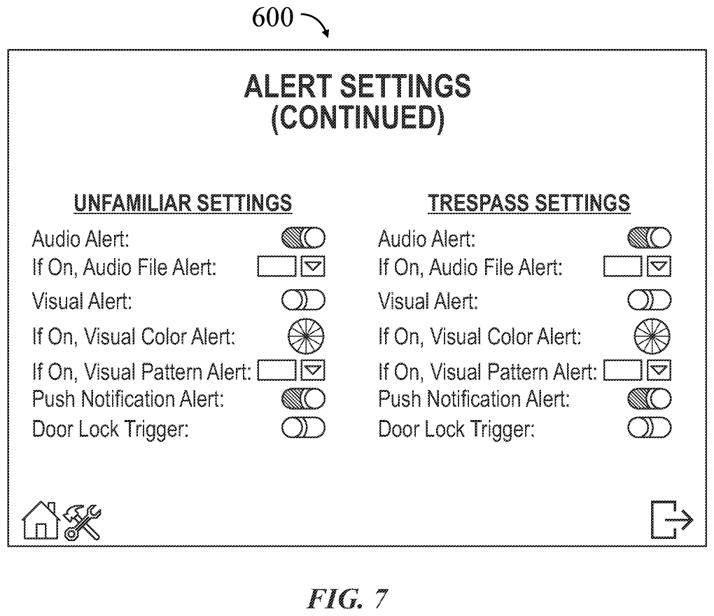

FIG. 6 and FIG. 7 illustrate a screenshots of the alert settings interface 600 wherein the user can input alert settings. The alert settings may be specific to a facility and/or to individuals associated with the facility. In such, alerts may be added or altered as needed by the user. In one example, the user may select a temperature threshold which, if exceeded, will trigger a response via the system (e.g., locking a door). The type of alert may be selected and may include audio and/or visual alerts. Alert responses may be adjusted depending on which security preference was breached. For example, the doors may be automatically locked if the individual's identity is not determined, but not locked if the individuals mask is not properly positioned on their face.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for a thermal monitoring security camera, the system comprising:
at least one camera positioned to monitor a facility;
an image processing module to receive imagery from the at least one camera and analyzes the imagery to determine, via a facial recognition module, an identity of an individual within the imagery and the presence of a mask on the individual's face;
at least one thermal camera to monitor the temperatures of each individual sensed by the thermal camera;
an alert module to transmit an alert if the temperature is above a threshold temperature; and
a security system to provide access or prevent access to the facility.

2. The system of claim 1, further comprising an application server in operable communication with a network, the application server configured to host an application system having a user interface for permitting a user to input a plurality of alert settings.

3. The system of claim 2, wherein the alert settings include a list of permitted individuals.

4. The system of claim 3, wherein the alert settings include the threshold temperature.

5. The system of claim 4, wherein the alert settings include a door lock trigger operated by the security system.

6. The system of claim 5, wherein the alert is audible.

7. The system of claim 6, wherein the alert is visible.

8. The system of claim 7, wherein the user inputs a face profile comprising an image utilized by the facial recognition module to compare the image to imagery captured by the camera.

9. The system of claim 8, further comprising a monitoring interface comprising a video feed and a thermal imagery feed.

10. The system of claim 9, wherein the monitoring interface comprises a listing of individuals identified by the facial recognition module.

11. A system for a thermal monitoring security camera, the system comprising:
at least one camera positioned to monitor a facility;
an image processing module to receive imagery from the at least one camera and analyze the imagery to determine, via a facial recognition module in communication with a comparator to compare the imagery with stored imagery in a facial profile database, an identity of an individual within the imagery and the presence of a mask on the individual's face;
at least one thermal camera to monitor the temperatures of each individual sensed by the thermal camera, wherein the temperature is compared to a threshold minimum temperature and a threshold maximum temperature to determine if each individual has an abnormal body temperature;
an alert module to transmit an alert if the temperature is above a threshold temperature; and
a security system to provide access or prevent access to the facility, the security system to permit or restrict access to the facility via one or more locking mechanisms located at one or more access points of the facility.

12. The system of claim 11, further comprising an application server in operable communication with a network, the application server configured to host an application system having a user interface for permitting a user to input a plurality of alert settings.

13. The system of claim 12, wherein the alert settings include a list of permitted individuals.

14. The system of claim 13, wherein the alert settings include the threshold temperature.

15. The system of claim 14, wherein the alert settings include a door lock trigger operated by the security system.

16. The system of claim 15, wherein the alert is audible.

17. The system of claim 16, wherein the alert is visible.

18. The system of claim 17, wherein the user inputs a face profile comprising an image utilized by the facial recognition module to compare the image to imagery captured by the camera.

19. The system of claim 18, further comprising a monitoring interface comprising a video feed and a thermal imagery feed.

20. The system of claim 19, wherein the monitoring interface comprises a listing of individuals identified by the facial recognition module.
